(19) United States
(12) Patent Application Publication
(10) Pub. No.: US 2006/0069416 A1
(43) Pub. Date: Mar. 30, 2006

(54) Retina implant for stimulating a retina as a function of incident light

(76) Inventors: Wilfried Nisch, Tuebingen (DE); Martin Stelzle, Reutlingen (DE); Heinz-Gerhard Graf, Magstadt (DE); Eberhart Zrenner, Tuebingen (DE); Markus Schubert, Tuebingen (DE); Hugo Haemmerle, Tuebingen (DE)

Correspondence Address: MORRISON & FOERSTER LLP, 12531 HIGH BLUFF DRIVE, SUITE 100, SAN DIEGO, CA, 92130-2040, US

(21) Appl. No.: 11/194,017

(22) Filed: Jul. 29, 2005

Related U.S. Application Data
(63) Continuation of application No. PCT/EP04/00768, filed on Jan. 29, 2004.

(30) Foreign Application Priority Data
Jan. 31, 2003 (DE) 103 04 831.6

(51) Int. Cl. A61N 1/18

(52) U.S. Cl. 607/54

(57) ABSTRACT

The present invention relates to a retina implant for the functional electrostimulation of a retina as a function of incident light. The retina implant has a stimulation chip that is designed to be implanted in the area of the retina of an eye. It further includes a radiation receiver that provides energy for the stimulation chip as a function of invisible radiation that occurs. In accordance with one aspect of the present invention, the radiation receiver is designed so as to be implanted in the area of the eye in a fashion spatially separated from the stimulation chip. The stimulation chip also has decoupling means in order to separate invisible scattered radiation from impinging visible light.

### RELATED APPLICATION

This is a continuation application of International Patent Application PCT/EP2004/000768, filed Jan. 29, 2004, designating the United States and published in German as WO 2004/067088 A1, which claims priority to German application number 103 04 831.6, filed Jan. 31, 2003.

### BACKGROUND OF THE INVENTION

### Field of the Invention

The present invention relates to a retina implant for the electrostimulation of a retina as a function of incident light,
having a stimulation chip that is designed to be implanted in the area of the retina of an eye, the stimulation chip including a multiplicity of contact points making contact with retina cells and a multiplicity of light-sensitive elements that drive the contact points as a function of impinging visible light, and
having a radiation receiver that provides energy for the stimulation chip as a function of impinging invisible radiation, preferably (infrared) IR radiation.

Such a retina implant is disclosed, for example, in DE 197 05 988 A1.

Attempts have been under way for some time to counteract a loss of vision owing to instances of retina degeneration with the aid of a retina implant of the above-named type. The fundamental idea is to give the patient an implant of a microelectronic stimulation chip in the area of the degenerated retina. The stimulation chip has a multiplicity of light-sensitive elements that generate electric pulses in the area of the retina as a function of the incident light. The retina implant can be placed on the retina (so called epiretinal approach), or it can be implanted in or under the retina (so called subretinal approach). The present invention is aimed chiefly at the practical implementation in the case of a subretinal implant, but it can also likewise be applied in the case of an epiretinal implant or other intraocular implants.

EP 0 460 320 A2 discloses a subretinal implant that is designed such that the impinging ambient light is intended to be already sufficient to generate the required stimuli for the retina cells. Details with reference to an additional energy supply of the implant are therefore not described.

DE 197 05 988 A1, mentioned at the outset, discloses a subretinal implant that is provided with a photovoltaic layer that is active for invisible electromagnetic radiation, the stimuli being switched locally by utilizing the voltage generated by the photovoltaic layer. This configuration is based on the idea of providing additional energy for the stimulation chip with the aid of electromagnetic radiation from the invisible spectral region, specifically infrared radiation. Consequently, sufficiently strong stimuli can be produced in the visible spectral region even in the event of weak lighting conditions.

The difficulty in the practical implementation consists in fashioning the implant such that it is not overdriven by the irradiation of the infrared radiation, that is to say in other words that it is rendered “blind” to the invisible infrared radiation. Said DE 197 05 988 A1 describes an approach in accordance with which the photovoltaic layer sensitive to infrared radiation is arranged below an amorphous layer that is only weakly doped, or not at all, and is transparent to the infrared radiation. On the other hand, the amorphous layer is sensitive to the visible light. During darkness, the conductivity of the amorphous layer is very low, that is to say it acts in principle as an insulator. If visible light is incident on the amorphous layer, the conductivity thereof is increased by a number of orders of magnitude. The charge carriers produced in the photovoltaic layer lying thereunder with the aid of the infrared radiation can then pass through the amorphous layer in order to stimulate the retina cells. The amorphous layer therefore acts as an electronic switch that is controlled by the visible light.

A further retina implant with additional incoupling of energy is disclosed in DE 199 31 083 A1. By contrast with the implant explained above, the incoupling of energy is, however, inductive here. The above outlined difficulties with incoupling infrared radiation are therefore eliminated. On the other hand, the inductive incoupling of additional energy requires an at least partially different structure of the retina implant. For example, according to the concept proposed in DE 199 31 083 A1, there is a need for a transformer device in order to transform the ac voltage induced on the side of the stimulation chip into a preferred dc voltage. The corresponding transformer device requires space in the area of the retina implant and is, however, also associated with a corresponding outlay on circuitry.

### SUMMARY OF THE INVENTION

In view of the above, it is an object of the present invention to specify an alternative for a retina implant in the case of which additional energy can be coupled in with the aid of electromagnetic radiation.

This object is achieved here by virtue of the fact that the radiation receiver is designed so as to be implanted in an area of the eye to be spatially separated from the stimulation chip, and that the stimulation chip has decoupling means in order to separate invisible scattered radiation from impinging visible light.

Otherwise than in the case of the retina implant disclosed in DE 197 05 988 A1, it is therefore proposed in accordance herewith to separate the radiation receiver and the actual stimulation chip from one another in order thus already to achieve a spatial decoupling of the two. For example, in a presently preferred embodiment it is proposed likewise to implant the radiation receiver in the region of the retina, but in a fashion offset from the stimulation chip. However, it is also possible as an alternative to this to arrange the radiation receiver outside on the eye, for example in the corner of the eye.

It is then possible on the basis of this measure to direct the invisible electromagnetic radiation, preferably infrared radiation, provided as additional energy, specifically onto the radiation receiver, or to keep it away from the light-sensitive elements of the stimulation chip. Given the selection of material and given the structural configuration of the light-sensitive elements and of the radiation receiver, this measure results in greater flexibility and breadth of configuration. The two components mentioned can therefore in each case be better optimized to their respective intended use, and this permits a higher efficiency. If the desired additional energy is radiated with the aid of IR laser diodes, for example, specifically onto the spatially offset radiation receiver, the undesired impairment of the light-sensitive elements in the stimulation chip can already be substantially reduced.

However, practical experiments with the spatially separate arrangement of stimulation chip and radiation receiver that is proposed here have shown that the spatial separation alone is not sufficient in order to exclude overdriving of the light-sensitive elements by the infrared radiation under all conditions. It has emerged, in particular, that scattered radiation components can reach the spatially offset stimulation chip because of instances of scattering, even when use is made of sharply focusing IR laser diodes. Problems are posed here both by scattered radiation components that reach the stimulation chip “directly”, and by (multiple) reflections of the IR radiation inside the eye. As a result of this, the present invention is based on the idea of supplementing the spatial separation of stimulation chip and radiation receiver by means of (further) decoupling means on the side of the stimulation chip. The combination of spatial separation and the (further) decoupling means permits overdriving of the light-sensitive elements to be reliably excluded.

The retina implant proposed here has in common with the retina implant disclosed in DE 197 05 988 A1 that additional energy is provided with the aid of invisible electromagnetic radiation that is radiated into the eye from outside. However, by contrast with the known retina implant, the present invention proposes a different path in order to remove the difficulties, arising in practice, with respect to the risk of overdriving the light-sensitive elements. The said object is thereby completely achieved.

According to another object of the invention, the stimulation chip includes as decoupling means a selective bandstop filter that suppresses the invisible electromagnetic radiation. The selective bandstop filter is preferably implemented in this case as an interference filter based on dielectric multiple alternating layers, in particular made from titanium dioxide and silicon dioxide.

With this embodiment, the disadvantageous infrared radiation on sides of the stimulation chip is specifically suppressed. The measure has the advantage that appropriate filtering technologies can be integrated very effectively into the production process of the stimulation chip. The use of the said materials further renders it possible to combine the desired filtering properties with measures for achieving biocompatibility, and this reduces the manufacturing outlay and thus the production costs.

According to a further object of the invention, the stimulation chip has as decoupling means light-sensitive elements of reduced sensitivity to the invisible radiation.

Whereas the aim with a bandstop filter is to keep the invisible scattered radiation away from the stimulation chip, this embodiment is based on the idea of “accepting” the presence of undesired scattered components of the invisible radiation and to make the light-sensitive elements insensitive thereto. There are various preferred possibilities for this purpose, and these are explained in more detail below. The measure is particularly advantageous as a supplement to a selective bandstop filter, since the various fundamental principles permit a particularly effective decoupling. Whereas with a selective bandstop filter of the type described above it is possible, for example, to achieve a decoupling in the range from two to three decimal decades (suppression by the factor 1/100th or 1/1000th), it is further possible to achieve one to four decades in addition with the aid of the measures described below.

In one embodiment of the abovementioned measure, the light-sensitive elements include a light-sensitive layer made from a material that has a bandgap of more than 1.5 eV between valence band and conduction band. Suitable materials are, for example, amorphous silicon with a high carbon doping, or else gallium arsenide, the amorphous silicon presently being preferred on the basis of its low toxicity.

Said measure is based on the finding that given such a material selection, impinging infrared radiation is incapable of conveying sufficiently many electrons from the valence band into the conduction band, for which reason given this material selection the light-sensitive elements are relatively insensitive to the infrared radiation. In other words, given this material selection the infrared radiation passes largely without effect through the light-sensitive elements. A decoupling in the range from approximately three to four decimal decades (factor 1/1,000th or 1/10,000th) can be achieved with this measure.

According to a still further object, the stimulation chip has a layered structure with a first layer near the surface and a second layer remote from the surface, the first layer near the surface including the light-sensitive elements and being thin in the direction of the incident light by comparison with a penetration depth of the invisible radiation.

This embodiment is based on the finding that because of its longer wavelength infrared radiation penetrates more deeply into a semiconductor material than, for example, does blue or green light from the visible spectral region. The fundamental idea here is thus to arrange the light-sensitive elements in an area near the surface of the stimulation chip in which the impinging infrared scattered radiation still does not develop an effect (not yet being absorbed). A decoupling as far as one decimal decade can be achieved by this measure. The flatter the first layer near the surface is in this case by comparison with the penetration depth of the invisible radiation, the more effective is this measure.

According to another object, the stimulation chip has as decoupling means a clock stage that activates the respective light-sensitive elements only for time periods.

This measure is based on the idea of decoupling the stimulation chip and the radiation receiver not spatially, but in addition also in time-dependent manner from one another. Given that the light-sensitive elements are activated in each case only for time periods, there is a possibility of irradiating infrared radiation into the inactive time periods for the purpose of supplying energy to the stimulation chip. A decoupling in the range from approximately two to four decimal decades can be achieved with the aid of this measure.

According to still another object of the invention, the radiation receiver has an antireflection layer which is selective for the invisible radiation and which is arranged preferably directly on its top side.

This measure is a further effective supplement to the measures already described above, since it already reduces the production of undesired interfering radiation in the invisible spectral region. However, since scattered components are reflected not only by the surface of the radiation receiver, but also are produced, for example, by refraction at the lens, this measure is chiefly preferred as a supplement to the previous measures. On the other hand, a further decoupling in the range of one decimal decade can be achieved according to the present finding.

It goes without saying that the features named above and those still to be explained below can be used not only in the combination respectively specified, but also in other combinations or on their own without departing from the scope of the present invention.

### BRIEF DESCRIPTION OF THE DRAWINGS

Embodiments of the invention are illustrated in the drawing and will be explained in more detail in the following description. In the drawing:

FIG. 1 shows a schematic of the novel retina implant in an eye,

FIG. 2 shows a first preferred embodiment of the novel retina implant,

FIG. 3 shows a second preferred embodiment of the novel retina implant, and

FIG. 4 shows a third preferred embodiment of the novel retina implant.

### DESCRIPTION OF PREFERRED EMBODIMENTS

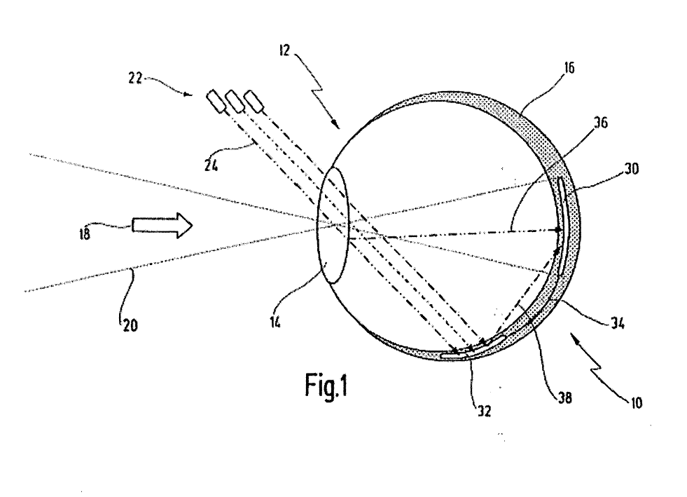

An embodiment of the novel retina implant is denoted in FIG. 1 in its entirety by the reference numeral 10.

The retina implant 10 is planted into the eye 12 of a patient, for the sake of simplicity only the lens 14 and the retina 16 of the eye are shown schematically. Visible light, indicated here schematically with the aid of an arrow 18, impinges into the eye through the lens 14 in accordance with the optical laws, which are sufficiently known. The beam path of the visible light 18 is shown here schematically with the reference numeral 20.

Reference numeral 22 denotes by way of example three IR laser diodes with the aid of which energy can be specifically coupled into the eye 12 in the form of IR radiation. The infrared radiation is denoted by reference numeral 24. The illustration with the aid of three IR laser diodes 22 is selected here by way of example, in order to show various beam paths. However, in practical execution it is preferred to provide only one IR laser diode 22. However, the invention is not limited to this, and can also be implemented with any other number of IR laser diodes and also with other suitable radiation sources.

In the embodiments shown here, the retina implant 10 in each case includes the actual stimulation chip 30 with the aid of which retina cells are stimulated as a function of the visible light 18, and a radiation receiver 32 offset therefrom. Stimulation chip 30 and radiation receiver 32 are electrically connected to one another, which is illustrated here by a line 34. The radiation receiver 32 operates as an energy transducer (photovoltaic element) and serves the purpose of receiving IR radiation 24 irradiated with the aid of the IR laser diodes 22, and of providing energy for the stimulation chip 30 as a function thereof.

A substantial decoupling between stimulation chip 30 and radiation receiver 32 is already achieved owing to the spatial separation of stimulation chip 30 and radiation receiver 32, and to the specific irradiation of the IR radiation onto the radiation receiver. Such a de-coupling is desirable in order to prevent overdriving of the light-sensitive elements present in the stimulation chip 30 (which is described below in more detail) by the IR radiation. However, as has emerged from practical experiments, the spatial separation alone is not sufficient in order to ensure an adequate decoupling under all conceivable operating conditions. It has emerged, in particular, that when the IR radiation 24 is irradiated into the eye 12 scattered components occur that can reach the stimulation chip 30 despite the spatial separation. The example of two important scattering sources is a scattered ray 36 that is produced by refraction when the IR radiation 24 emerges from the lens 14. A further scattered ray 38 shows reflections at the surface of the radiation receiver 32. Moreover, multiple reflections can occur inside the vitreous body of the eye 12, and so the stimulation chip 30 is exposed to IR scattered radiation from various directions for a variety of causes. According to the invention, the stimulation chip 30 is therefore provided with decoupling means that permit the invisible scattered radiation (IR radiation) 24 to be separated from impinging visible light 18.

In the following description of preferred embodiments, identical reference numerals in each case denote the same elements as in FIG. 1.

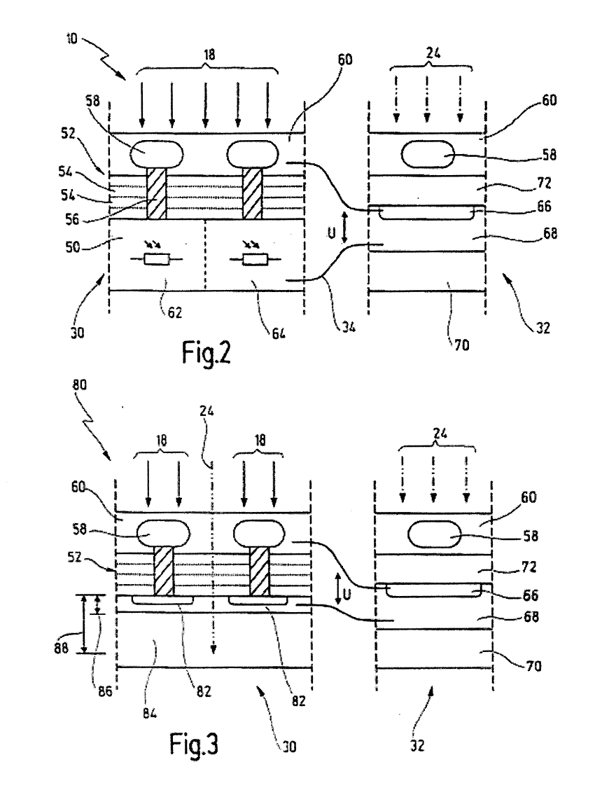

FIG. 2 shows a first embodiment of the retina implant 10. The schematic design of the stimulation chip 30 and its position inside the retina 16 is shown in the left-hand part of FIG. 2 in a detail. The right-hand part of FIG. 2 shows the schematic design of the radiation receiver 32, which is likewise implanted in the retina 16 in a fashion spatially offset from the stimulation chip 30.

The stimulation chip 30 here includes a light-sensitive layer 50 made from a semiconductor material that has a bandgap between valence band and conduction band (not illustrated here) of at least 1.5 eV. In a preferred embodiment, this is amorphous silicon with a high carbon doping. In this preferred embodiment, the bandgap is approximately 2 eV. Alternatively, it would also be possible to use gallium arsenide, something which is, however, less preferable because of its toxic effect, or any other material that has the bandgap mentioned.

Arranged above the light-sensitive layer 50 is a layer 52 that is constructed from a number of partial layers 54. The material and the thickness of the partial layers 54 are selected such that the layer 52 acts as an interference filter that specifically suppresses impinging scattered components of the invisible IR radiation 24. The partial layers 54 are preferably produced in an alternating fashion from titanium dioxide and silicon dioxide, since these materials have a good biocompatibility. However, it is also possible to use other suitable materials and, moreover, also other filter types. Moreover, embodiments that manage without a layer 52 can also be implemented.

The reference numeral 56 denotes an electrode that connects a section of the light-sensitive layer 50 to a retina cell 58 to be stimulated. The retina cell 58 is illustrated here schematically as part of a layer of tissue 60 that is a constituent of the retina 16.

A multiplicity of electrodes 56 are provided in the embodiment shown in order to make contact with a multiplicity of retina cells 58 in a spatially resolved fashion. Each electrode 56 is connected to a section 62, 64 of the light-sensitive layer 50. The individual sections 62, 64 are therefore the light-sensitive elements by means of which the retina cells 58 are stimulated as a function of incident visible light 18.

The light-sensitive elements (sections) 62, 64 are implemented in this embodiment as photoresistors that vary their conductivity upon impingement of visible light 18. As an alternative thereto, however, it would also be possible to implement photodiodes, photo-transistors and other elements acting as light-controlled switches inside the layer 50.

In a way known per se, the radiation receiver 32 includes a pn junction; that is to say a structure that is illustrated here by way of example with a p doped layer 66 and an n doped layer 68 lying thereunder. The pn junction of the two layers 66, 68 is optimized with regard to its use as photovoltaic element (dimension, doping of the layers etc.). The pn junction is formed on a substrate 70 in a way known per se. An antireflection layer 72 transparent to the IR radiation 24 is arranged above the p layer 66. The proportion of the scattered radiation (scattered ray 38 in FIG. 1) is reduced owing to this antireflection layer 72, something which contributes to a further improved decoupling of the stimulation chip 30.

As already mentioned, the pn junction of the layers 66, 68 operates here as a photovoltaic element tuned to the infrared radiation 24. Consequently, upon impingement of the infrared radiation 24 electric voltage U that is used for stimulating the retina cells 58 is produced in the area of the space charge zone (not shown here) of the pn junction. The voltage U generated is consequently fed to the stimulation chip 30 via the line 34.

In the case of the stimulation chip 30, the material of the light-sensitive layer 50 acts as an important decoupling element. Specifically, because of the high bandgap the relatively long wave infrared radiation is not capable of transporting electrons in appreciable quantity from the valence band into the conduction band. Consequently, impinging IR scattered radiation 36, 38 scarcely varies the conductivity of the light-sensitive elements 62, 64. However, the light-sensitive elements 62, 64 have their conductivity controlled by impinging visible light 18 and can consequently stimulate the retina cells 58 with the aid of the applied voltage U as a function of the visible light.

A further embodiment of the retina implant is denoted in FIG. 3 in its entirety by reference numeral 80. The radiation receiver 32 is designed in this case exactly as in the case of the embodiment in accordance with FIG. 2. Furthermore, the retina implant 80 in turn advantageously has a selective bandstop filter, in the form of the interference filter 52 here.

However, by contrast with the embodiment in accordance with FIG. 2, here the stimulation chip 30 has as light-sensitive element a multiplicity of photodiodes of which the pn junction 82 is shown symbolically in each case. The photodiodes 82 are formed in a way known per se on a substrate 84. It is provided here as a substantial decoupling means from IR scattered radiation that the thickness 86 of the light-sensitive pn junction 82 is small by comparison with the penetration depth 88 of the IR scattered radiation 24. Since, because of its comparatively long wavelength, the IR scattered radiation penetrates more deeply into the semiconductor material of the stimulation chip 30 than does, for example, visible light in the blue or green spectral region, good decoupling of the stimulation chip 30 from the IR scattered radiation can be achieved by means of the described dimensioning and arrangement of the light-sensitive elements.

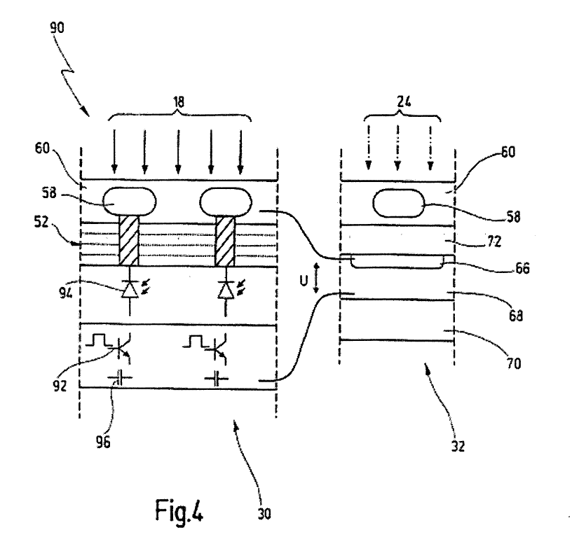

A further embodiment of the retina implant is denoted in its entirety in FIG. 4 by the reference numeral 90. Identical reference numerals denote the same elements as before.

Here, the stimulation chip 30 has as decoupling means a multiplicity of clock stages 92 with the aid of which the light-sensitive elements (here photodiodes 94) are respectively activated only in time periods. Such an arrangement permits the IR radiation 24 to be coupled in each case into the eye 12 in the time periods in which the light-sensitive elements 94 are inactive. In other words, the incoupling of the IR energy is performed in this case in pulses, specifically in the time periods in which the light-sensitive elements 94 are inactive. In order to hold the irradiated energy ready for stimulating the retina cells 58, storage elements are further provided in this case, here in the form of capacitors 96, for example. Moreover, the retina implant 90 in turn has the interference filter 52 as further decoupling means.

It goes without saying that combinations of the decoupling means illustrated here with the aid of the individual embodiments are also possible in a form not explicitly shown here, in order to achieve an even higher decoupling, if appropriate.

Experimental investigations on a model apparatus, dead pig eyes as well as living and dead rabbit eyes returned the following results:

In order to ensure an adequate power supply for the stimulation chip 30, an optical power of 15 mW must be irradiated onto the radiation receiver 32. In the case of an arrangement as shown in FIG. 1, because of various reflections IR scattered radiation 36, 38 of the order of magnitude of 0.09 mW/cm2 falls onto the stimulation chip 30. This means that approximately 1/2000 of the irradiated IR radiation falls onto the stimulation chip 30.

0.09 mW/cm2 corresponds approximately to the situation when a surface illuminated by daylight, for example a page of a book, is imaged onto the stimulation chip 30.

Thus, the spatial separation of the stimulation chip 30 and radiation receiver 32 yields a strong suppression of scattered light that, however, will not lead to satisfactory visual results in many cases, for example given a brightness that is lower than daylight.

A further suppression of scattered light by two to three orders of magnitude can be achieved by the use of a selective bandstop filter 52, for example an edge filter, with an antireflection layer 32.

A suitable selection of the bandgap yields a further suppression of scattered light of one to two orders of magnitude, it being possible to improve the suppression of scattered light by at least one further order of magnitude by the temporal separation.

1. A retina implant for stimulating retina cells within a retina of an eye, as a function of incident light, said retina implant comprising
a stimulation chip that is designed to be implanted in an area of the retina, said stimulation chip including a multiplicity of contact points making contact with said retina cells, and a multiplicity of light-sensitive elements connected to said contact points for stimulating said retina cells as a function of impinging visible light, and
a radiation receiver that provides energy for said stimulation chip as a function of impinging invisible radiation,
wherein said radiation receiver is designed so as to be implanted in an area of the eye to be spatially separated from said stimulation chip, and
wherein said stimulation chip is provided with decoupling means in order to separate invisible scattered radiation from impinging visible light.

2. The retina implant of claim 1, wherein said decoupling means comprise a selective bandstop filter that suppresses impinging invisible radiation.

3. The retina implant of claim 1, wherein said light-sensitive elements have reduced sensitivity to the invisible radiation.

4. The retina implant of claim 2, wherein said light-sensitive elements have reduced sensitivity to the invisible radiation.

5. The retina implant of claim 3, wherein said light-sensitive elements include a light-sensitive layer made from a material having a bandgap of more than 1.5 eV between valence band and conduction band.

6. The retina implant of claim 4, wherein said light-sensitive elements include a light-sensitive layer made from a material having a bandgap of more than 1.5 eV between valence band and conduction band.

7. The retina implant of claim 3, wherein said stimulation chip has a layered structure with a first layer near a surface of said stimulation chip and a second layer remote from said surface, the first layer near said surface including said light-sensitive elements and being thin in the direction of the incident light as compared with a penetration depth of said invisible radiation.

8. The retina implant of claim 5, wherein said stimulation chip has a layered structure with a first layer near a surface of said stimulation chip and a second layer remote from said surface, said first layer near said surface including said light-sensitive elements and being thin in the direction of the incident light as compared with a penetration depth of said invisible radiation.

9. The retina implant of claim 1, wherein said decoupling means comprises a clock stage that activates said respective light-sensitive elements only for time periods.

10. The retina implant of claim 1, wherein said radiation receiver comprises an antire-flection layer for said invisible radiation.

11. The retina implant of claim 1, wherein said invisible radiation is IR radiation.

12. A retina implant for stimulating retina cells within a retina of an eye, as a function of incident light, said retina implant comprising
a stimulation chip that is designed to be implanted in an area of the retina, said stimulation chip including a multiplicity of contact points making contact with said retina cells, and a multiplicity of light-sensitive elements connected to said contact points for stimulating said retina cells as a function of impinging visible light, and
a radiation receiver that provides energy for said stimulation chip as a function of impinging IR radiation,
wherein said radiation receiver is designed so as to be implanted in an area of the eye to be spatially separated from said stimulation chip, and
said stimulation chip comprises a selective bandstop filter for separating invisible scattered radiation from impinging visible light by suppressing said IR radiation.

13. A retina implant for stimulating retina cells within a retina of an eye, as a function of incident light, said retina implant comprising
a stimulation chip that is designed to be implanted in an area of the retina, said stimulation chip including a multiplicity of contact points making contact with said retina cells, and a multiplicity of light-sensitive elements connected to said contact points for stimulating said retina cells as a function of impinging visible light, and
a radiation receiver that provides energy for said stimulation chip as a function of impinging IR radiation,
wherein said radiation receiver is designed so as to be implanted in an area of the eye to be spatially separated from said stimulation chip, and
wherein said light-sensitive elements have reduced sensitivity to said IR radiation.

14. A retina implant for stimulating retina cells within a retina of an eye, as a function of incident light, said retina implant comprising
a stimulation chip that is designed to be implanted in an area of the retina, said stimulation chip including a multiplicity of contact points making contact with said retina cells, and a multiplicity of light-sensitive elements connected to said contact points for stimulating said retina cells as a function of impinging visible light, and
a radiation receiver that provides energy for said stimulation chip as a function of impinging IR radiation,
wherein said radiation receiver is designed so as to be implanted in an area of the eye to be spatially separated from said stimulation chip, and
wherein said stimulation chip comprises a clock stage that activates said light-sensitive elements only for certain time periods.

15. A retina implant for stimulating retina cells within a retina of an eye, as a function of incident light, said retina implant comprising
a stimulation chip that is designed to be implanted in an area of the retina, said stimulation chip including a multiplicity of contact points making contact with said retina cells, and a multiplicity of light-sensitive elements connected to said contact points for stimulating said retina cells as a function of impinging visible light, and
a radiation receiver that provides energy for said stimulation chip as a function of impinging IR radiation,
wherein said radiation receiver is designed so as to be implanted in an area of the eye to be spatially separated from said stimulation chip, and
said stimulation chip comprises a selective bandstop filter for separating invisible scattered radiation from impinging visible light by suppressing said IR radiation,
wherein said stimulation chip comprises a clock stage that activates said light-sensitive elements only for certain time periods.

16. A retina implant for stimulating retina cells within a retina of an eye, as a function of incident light, said retina implant comprising
a stimulation chip that is designed to be implanted in an area of the retina, said stimulation chip including a multiplicity of contact points making contact with said retina cells, and a multiplicity of light-sensitive elements connected to said contact points for stimulating said retina cells as a function of impinging visible light, and
a radiation receiver that provides energy for said stimulation chip as a function of impinging IR radiation,
wherein said radiation receiver is designed so as to be implanted in an area of the eye to be spatially separated from said stimulation chip, and
said stimulation chip comprises a selective bandstop filter for separating invisible scattered radiation from impinging visible light by suppressing said IR radiation,
wherein said stimulation chip comprises a clock stage that activates said light-sensitive elements only for certain time periods,
wherein said light-sensitive elements have reduced sensitivity to said IR radiation.
